(19) United States
(12) Patent Application Publication
(10) Pub. No.: US 2025/0026777 A1
(43) Pub. Date: Jan. 23, 2025

(54) COCRYSTALS OF NICOTINAMIDE RIBOSIDE SALTS, CRYSTALLINE FORMS, METHODS OF PREPARATION, AND APPLICATIONS THEREOF

(71) Applicant: Beiture LLP, Newton, MA (US)

(72) Inventors: Dedong Wu, Newton, MA (US); Branden Z. Wu, Newton, MA (US)

(73) Assignee: Beiture LLP, Newton, MA (US)

(21) Appl. No.: 18/352,410

(22) Filed: Jul. 14, 2023

(51) Int. Cl. C07H 19/048; C07C 37/84; C07C 39/21

(52) U.S. Cl. CPC C07H 19/048; C07C 37/84; C07C 39/21; C07B 2200/13

(57) ABSTRACT

The present disclosure relates to cocrystals of nicotinamide riboside salts (NR), which can be used in preparing pharmaceutical or nutraceutical compositions for longevity or for the treatment or prevention of cardiovascular diseases, central nervous system diseases, aging, or other conditions. Specifically, the present disclosure relates to the cocrystal of a nicotinamide riboside salt, including but not limited to, the resveratrol cocrystal of nicotinamide riboside chloride, its polymorphic crystalline forms, methods of preparation, and applications thereof.

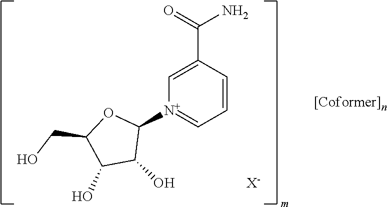

wherein X⁻is a negatively charged anion, the Coformer (abbreviation of cocrystal former) is a neutral compound or a neutral form of an acid or base, m and n are integers larger than 1.

### BACKGROUND OF THE INVENTION

Nicotinamide riboside (IUPAC name: 3-Carbamoyl-1-[(2R,3R,4S,5R)-3,4-dihydroxy-5-(hydroxymethyl) oxolan-2-yl] pyridin-1-ium) is a naturally occurring pyridine-nucleoside presenting in almost all living organisms and is a form of vitamin B3. Nicotinamide riboside (NR) functions as a precursor to nicotinamide adenine dinucleotide or NAD+, which is an essential coenzyme that plays important roles in various metabolic pathways. The overall content increasement of NAD+ has been confirmed as a valuable strategy for treating and/or preventing a wide variety of pathophysiological conditions. Thus, nicotinamide riboside (NR) has been demonstrated as a promising candidate for preventing and/or treating several conditions including cardiovascular, metabolic, and neurodegenerative disorders (Nutrients 2020, 12, 1616; doi.org/10.3390/nu12061616). Nicotinamide riboside is marketed as nicotinamide riboside chloride salt (NR—Cl), which is generally recognized as safe (GRAS) by the FDA (U.S. Food and Drug Administration) and is widely commercialized as food supplements for anti-aging and longevity. Nicotinamide riboside chloride (NR—Cl) has a structure depicted below.

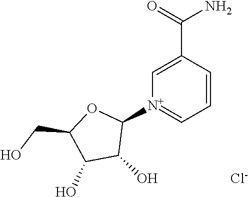 [SMILES: NC(=O)c1ccc[n+]([C@@H]2O[C@H](CO)[C@@H](O)[C@H]2O)c1 ; [Cl-]]

Various crystalline forms of nicotinamide riboside chloride (NR—Cl) have been discovered and disclosed in several patents in recent years in order to improve its pharmaceutical properties, including chemical purity, chemical stability, physicochemical property, powder property, et al. (WO2015186068, WO2016014927, WO2016144660, WO2018089830, WO2019126482, CN111808156). Crystalline Form B of NR—Cl was found to be the most stable among all the three identified crystalline forms (J. Chem. Crystallogr. 2023, online on May 25, doi.org/10.1007/s10870-023-00985-1). Recently, other pharmaceutically or nutraceutical acceptable crystalline salts, e.g., NR hydrogen tartrate salt, NR hydrogen malate salt, have also been developed (WO2021013795).

Resveratrol (herein abbreviated as RSV, 3,5,4′-trihydroxy-trans-stilbene, IUPAC name: 5-[(E)-2-(4-Hydroxyphenyl) then-1-yl]benzene-1,3-diol) is a stilbenoid, a type of natural phenol, and a phytoalexin produced by several plants in response to injury or when the plant is under attack by pathogens, such as bacteria or fungi. Sources of resveratrol in food include the skin of grapes, blueberries, raspberries, mulberries, and peanuts. As commonly used as a dietary supplement and studied in laboratory models of human diseases, resveratrol has been studied for lifespan improvement and its potential therapeutic uses in various therapeutical areas, including cardiovascular disease, cancer, metabolic syndrome, cognition, diabetes and other conditions. Resveratrol shows a low bioavailability, likely due to the poor aqueous solubility and rapid metabolism of the molecule, limiting its clinical efficacy. Various strategies attempted to improve resveratrol effectiveness both for systemic and for topical administration. Resveratrol has a structure depicted below.

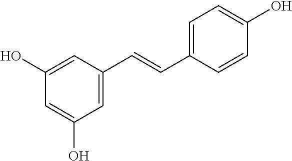 [SMILES: Oc1ccc(/C=C/c2cc(O)cc(O)c2)cc1]

Scientific study has suggested that the NAD+ precursor, e.g., nicotinamide riboside (NR) or nicotinamide mononucleotide (NMN), combined with resveratrol could have increased the levels of NAD+ in the heart and muscle. (Pharmacol. Res. Perspect. 2022, 10:e00986. doi.org/10.1002/prp2.986). Accordingly, there is a need to develop a novel crystalline form containing both nicotinamide riboside and resveratrol to provide a new chemical entity with improved pharmaceutical properties, e.g., higher chemical purity for drug product quality, better hygroscopicity for processing and storage, or better chemical stability for longer shelf life, for pharmaceutical or nutraceutical products.

Other objects, advantages and features of the present disclosure will become apparent from the following specification taken in conjunction with the accompanying examples or drawings.

### BRIEF SUMMARY OF THE INVENTION

Provided herein is a cocrystal of nicotinamide riboside salt, which can be used in a pharmaceutical or nutraceutical composition for treatment or prevention of diseases or conditions in a human or an animal in need thereof.

In one aspect, the disclosure provides a compound of Formula I:

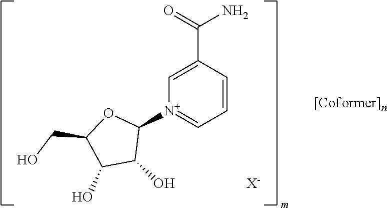

wherein X⁻is a negatively charged anion, the Coformer (abbreviation of cocrystal former) is a neutral compound or a neutral form of an acid or base, m and n are integers larger than 1.

In some embodiments, the compound of Formula I is selected from the group consisting of:

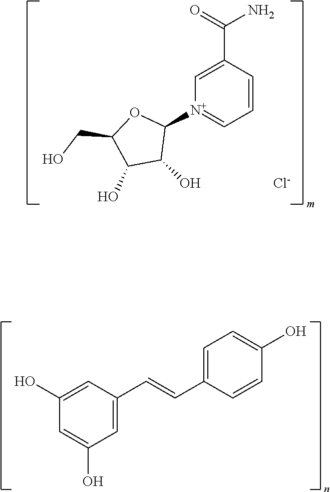

wherein X⁻is a chloride anion, the Coformer is resveratrol, m and n are integers larger than 1.

In some embodiments, the compound of Formula I is selected from the group consisting of:

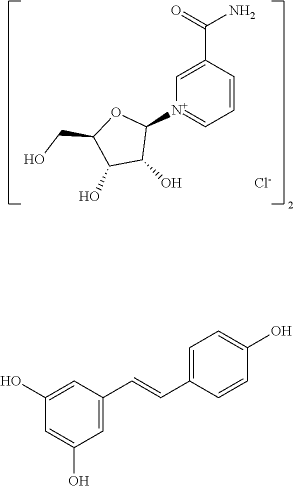

wherein X⁻is a chloride anion, the Coformer is resveratrol, m=2, n=1, which is abbreviated as [NR—Cl]₂[RSV] cocrystal.

The chemical composition is characterized and confirmed by nuclear magnetic resonance (NMR) spectroscopy. Remarkably, the resveratrol (RSV) cocrystal of nicotinamide riboside chloride (NR—Cl) crystallizes in an anhydrous form or a solvated form with a 2:1 molar ratio of NR—Cl/RSV.

In another aspect, provided herein are crystalline forms of the resveratrol cocrystal of nicotinamide riboside chloride ([NR—Cl]₂[RSV] cocrystal). Crystalline forms are characterized by powder X-ray diffraction (PXRD) method.

In one embodiment as disclosed herein, the exemplary crystalline form of [NR—Cl]₂[RSV] cocrystal is an anhydrous form, designated as Form A, and is characterized by a PXRD pattern comprising one or more peaks expressed as 2θ±0.2° of about 5.6°, about 11.0°, about 11.3°, about 12.3°, about 13.7°, about 14.0°, about 15.9°, about 19.8°, about 26.0°, and about 26.4°.

Surprisingly, Form A of [NR—Cl]₂[RSV] cocrystal ([NR—Cl]₂[RSV]-Form A) shows a higher melting/decomposition temperature and a larger value of heat of fusion compared to nicotinamide riboside chloride (NR—Cl) crystalline forms, including anhydrous Form A and Form B, suggesting a better thermal stability. [NR—Cl]₂[RSV] cocrystal Form A also shows a better hygroscopicity compared to nicotinamide riboside chloride (NR—Cl) crystalline forms, including both NR—Cl-Form A and NR—Cl-Form B.

In one embodiment as disclosed herein, the exemplary crystalline form of [NR—Cl]₂[RSV] cocrystal is a solvated form, designated as Form B, and is characterized by a PXRD pattern comprising one or more peaks expressed as 2θ±0.2° of about 5.5°, about 8.2°, about 11.0°, about 12.1°, about 12.9°, about 13.5°, about 15.3°, about 19.6°, about 22.0°, and about 27.0°.

Surprisingly, Form B of [NR—Cl]₂[RSV] cocrystal ([NR—Cl]₂[RSV]-Form B) shows a better hygroscopicity compared to nicotinamide riboside chloride (NR—Cl) crystalline forms, including both NR—Cl-Form A and NR—Cl-Form B.

In another aspect, provided herein is a method to prepare the desired cocrystal of nicotinamide riboside salt. The general reaction disclosed herein to prepare the [NR—Cl]₂[RSV] cocrystal by a reaction crystallization process is shown as the following scheme:

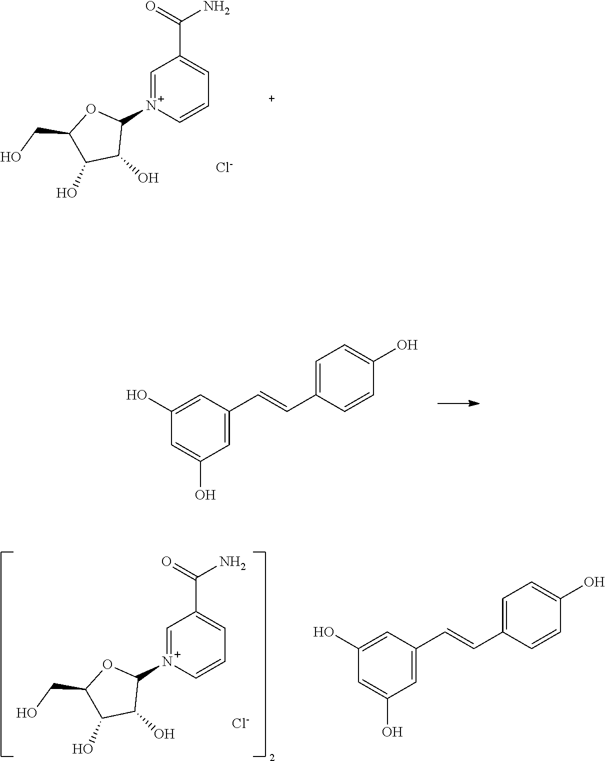

In one embodiment as disclosed herein, the disclosed method uses recrystallization process to prepare Form A of [NR—Cl]₂[RSV] cocrystal by the procedures. 1) Dissolving the starting materials of NR—Cl and RSV in MeOH, MeOH/H₂O, EtOH/H₂O or IPA/H₂O. 2) Concentrating the resulting solution in a vacuum to dryness. 3) Stirring the resulting solid in dry EtOH solvent. 4) Isolating the resulting crystalline Form A of [NR—Cl]₂[RSV] by filtration, washing, and drying.

In one embodiment as disclosed herein, the disclosed method uses adding anti-solvent process to prepare Form B of [NR—Cl]₂[RSV] cocrystal by the procedures. 1) Dissolving the starting materials of NR—Cl and RSV in MeOH/H₂O, EtOH/H₂O or IPA/H₂O. 2) Adding anti-solvent of IPA into the solution and stirring to produce a homogenous slurry. 3) Isolating the resulting crystalline Form B of [NR—Cl]₂[RSV] by filtration, washing, and drying.

In one embodiment as disclosed herein, the disclosed method uses slurry process to prepare Form B of [NR—Cl]₂[RSV] cocrystal by the procedures. 1) Suspending the starting materials of NR—Cl and RSV in a minimum amount of EtOH/H₂O co-solvent. 2) Stirring the suspension until the starting physical mixture fully converted cocrystal to yield a wet-cake. 3) Adding EtOH into the resulting wet-cake and stirring to produce a homogenous slurry. 4) Isolating the resulting crystalline Form B of [NR—Cl]₂[RSV] by filtration, washing, and drying.

In another aspect, the disclosure provides use of a compound of Formula I in a composition of a pharmaceutical or nutraceutical product.

In another aspect, the disclosure provides use of a compound of Formula I for the treatment or prevention of a cardiovascular disease, aging, or other conditions in a human or an animal in need thereof.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows the 1H NMR spectrum of Form A of the resveratrol cocrystal of nicotinamide riboside chloride.

FIG. 2 shows the powder X-ray diffraction pattern of Form A of the resveratrol cocrystal of nicotinamide riboside chloride.

FIG. 3 shows the DSC diagram of Form A of the resveratrol cocrystal of nicotinamide riboside chloride.

FIG. 4 shows the TGA diagram of Form A of the resveratrol cocrystal of nicotinamide riboside chloride.

FIG. 5 shows the 1H NMR spectrum of Form B of the resveratrol cocrystal of nicotinamide riboside chloride.

FIG. 6 shows the powder X-ray diffraction pattern of Form B of the resveratrol cocrystal of nicotinamide riboside chloride.

FIG. 7 shows the DSC diagram of Form B of the resveratrol cocrystal of nicotinamide riboside chloride.

FIG. 8 shows the TGA diagram of Form B of the resveratrol cocrystal of nicotinamide riboside chloride.

### DETAILED DESCRIPTION OF THE INVENTION

### Field of the Invention

The present disclosure relates to a pharmaceutical cocrystal of nicotinamide riboside salt, specifically, a resveratrol cocrystal of nicotinamide riboside chloride, which can be used in a pharmaceutical or nutraceutical composition for the treatment or prevention of diseases or conditions in a human or an animal in need thereof.

### Definitions

The embodiments of this disclosure are not limited to any compositions and methods, which can vary and are understood by skilled artisans. So that the present disclosure may be more readily understood, certain terms are defined. Unless defined otherwise, all technical and scientific terms used herein have the same meaning as commonly understood by one of ordinary skills in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below. Unless stated otherwise, the following terms and phrases as used herein are intended to have the following meanings:
The term “solid form”, “solid”, or related terms, as used herein, refers to a physical form that is not predominantly in a liquid or a gaseous state. A solid form may be crystalline, amorphous, or a mixture thereof.

The term “amorphous,” “amorphous form,” or related terms refers to a substance, component, or product that is not crystalline as determined by X-ray diffraction, solid NMR, or other analytical techniques as known by one skilled in the art. In particular, the term “amorphous form” describes a disordered solid form, e.g., a solid form lacking long range crystalline order.

The term “crystalline”, “crystalline solid”, “crystal solid”, “crystal form”, “crystalline form”, or related terms, as used herein, refers to any solid substance, material, compound, a mixture of compounds, or product exhibiting three-dimensional order, which contrasts with an amorphous solid substance, giving a distinctive PXRD pattern or solid NMR spectrum with sharply defined peaks.

The terms “polymorphs,” “polymorphic forms”, or related terms refer to a crystalline solid comprising two or more crystal forms of the same molecule, mixtures of molecules, salt, co-crystal, or combination thereof. The term “anhydrous form” or “neat form” refers to a solid form of a substance which contains only compound molecule in the solid composition. The term “solvate” or “solvated form” refers to the existence of a crystalline form for a particular solvate composition. Similarly, “hydrate” or “hydrate form” refers to the existence of a crystalline form for a particular hydrate composition. The term “desolvated solvate” refers to a crystal form of a substance which may be prepared by removing the solvent from a solvate. A crystalline solid can be a material containing a single crystalline form or multiple crystalline forms.

In some embodiments, a crystalline solid is crystalline as determined, e.g., by XRPD, solid NMR, Raman spectroscopy, polarized light microscopy (PLM), thermal analysis, and/or moisture absorption analysis. In some embodiments, a crystal form of a substance may be “chemically pure,” e.g., contains less than about 1%, about 2%, about 3%, about 4%, about 5%, about 6%, about 7%, about 8%, about 9%, about 10%, about 15%, about 20%, about 25%, about 30%, about 35%, or about 40% of other chemical substances on a weight basis. In some embodiments, a crystalline solid of a substance may be “physically pure,” e.g., contains less than about 5%, about 10%, about 15%, about 20%, about 25%, about 30%, about 35%, or about 40% of other crystal forms or amorphous forms on a weight basis. A “salt”, as used herein, refers to a solid comprising both anions and cations in equal molar amount. A salt can be a “single-component” solid. A salt can also be a “multiple-component” solid comprising one or more additional species, such as nonionic molecules, cocrystal molecules, and/or solvent molecules. For example, a crystalline multiple-component salt further comprises one or more non-covalently bonded species at regular positions in its crystal lattice.

A “cocrystal” or “pharmaceutical cocrystal”, as used herein, refers to a single crystalline material consisting of at least two molecular components as solids at room temperature and present in a definite stoichiometric ratio. It usually comprises at least two neutral components (compounds) co-existing in crystal lattice without covalent bonds between the components. In some cases, a salt can also form a cocrystal with a neutral compound or an acid/base in its neutral state. The ratio between these components can be measured by single crystal structure analysis, NMR or other techniques.

“Crystallization” is the process of atoms or molecules arranging into a well-defined crystal lattice in order to minimize their energetic state, which is known as a crystal. The method to make crystal is known as crystallization process or crystal formation. Attributes of the crystallization process depend largely on the chemical structure of a compound and other factors such as temperature, pressure, and time of process. Crystallization is also a chemical solid-liquid separation technique, in which mass transfer of a solute from the liquid solution to a pure solid crystalline phase occurs. Crystal formation can be achieved by various methods, such as: heating/cooling, evaporation, addition of a second solvent to reduce the solubility of the solute (technique known as antisolvent or drown-out), solvent layering, sublimation, changing the cation or anion, as well as other methods. The process with certain crystallization conditions does not guarantee crystal formation or a solid with acceptable crystallinity. Sometimes, the repeating crystallization process to increase the chemical purity or the crystallinity is also known as “recrystallization”.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “therapeutically effective amount”, as used herein, is the amount of compound of the active pharmaceutical ingredient presenting in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend upon numerous factors, for example the particular compound structure, the specific activity of the composition, the delivery device employed, the physical characteristics of the composition, its intended use, as well as patient considerations such as severity of the disease state, patient cooperation, etc., and can readily be determined by one skilled in the art based upon the information provided herein.

### Cocrystal Compounds

Provided herein is a cocrystal of nicotinamide riboside salt, which can be used in a pharmaceutical or nutraceutical composition for treatment or prevention of diseases or conditions in a human or an animal in need thereof.

As a permanently positively charged cation, nicotinamide riboside is not able to form a conventional cocrystal directly with other component or components containing only neutral molecule or acid/base in its neutral state, innovative cocrystals of the nicotinamide riboside salt, which contains a negatively charged anion to balance the charge, have been proposed, designed, tested, and analyzed to discover and develop new chemical entities of nicotinamide riboside used for pharmaceutical and nutraceutical composition.

In one aspect, the disclosure provides a compound of Formula I:

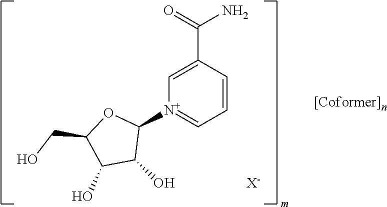

wherein X⁻is a negatively charged anion, the Coformer is a neutral compound or a neutral form of an acid or base, m and n are integers larger than 1, m and n are integers larger than 1.

The negatively charged anion X⁻is a pharmaceutical acceptable counter ion, which includes, but is not limited to, chloride, bromide, sulfate, phosphate, methyl sulfonate, acetate, malate, tartrate, citrate, fumarate, lactate, maleate, mandelate, malonate, succinate, ascorbate, gluconate, benzoate, besylate, tosylate, hippurate, camsylate, napasylate, napadisylate, stearate, undecylenate, and other acidic counterions, which have been used in pharmaceutical or nutraceutical products. The cocrystal former or conformer can be either a neutral compound or acid/base in its neutral state, which is selected from any drug excipients included in the GRAS (Generally Recognized as Safe) list published by the FDA.

Negatively charged anions as acidic counterions have been screened and selected to balance the permanently positively charged nicotinamide riboside. At the same time, neutral molecules or acids/bases in their neutral states have also been screened and selected to achieve forming pharmaceutical acceptable cocrystals.

In some embodiments, the compound of Formula I is selected from the group consisting of:

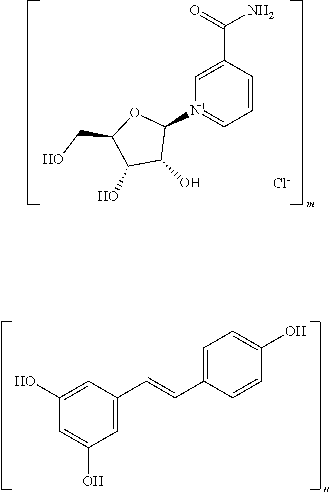

wherein X⁻is the chloride anion, the Coformer is resveratrol, m and n are integers larger than 1.

Nicotinamide riboside is marketed as nicotinamide riboside chloride salt (NR—Cl) and is widely commercialized in food supplements for anti-aging and longevity. Three crystalline forms of NR—Cl have been identified, and the two anhydrous forms are found in the nicotinamide riboside commercial products. Despite improvement of chemical stability and physical property compared to the amorphous material, NR—Cl crystalline compound still possesses some restrictions in commercial products, for example the storage, due its non-optimal physicochemical property. All the current marketed nicotinamide riboside products were suggested to be stored in lower temperature and avoid the high humidity due to thermal instability and hygroscopicity issues of the NR—Cl substance. It is desirable to discover and develop a novel crystalline form with improved pharmaceutical properties. The approach of forming a novel cocrystal of the nicotinamide riboside chloride with suitable cocrystal formers has been investigated and disclosed herein. The suitable cocrystal formers were chosen from polyphenols, naturally occurring highly powerful antioxidants, including phenolic acids, flavonoids, stilbenes, and lignans. Among these polyphenols, resveratrol, pterostilbene, quercetin or fisetin is often combined with the nicotinamide riboside product for synergic therapeutical effect. Various methods have been experimentally screened and tested to identify suitable cocrystal of nicotinamide riboside salt because there are not any predictive techniques or tools to guarantee the formation of co-crystals of the nicotinamide riboside salt with a list of coformers.

Surprisingly, a new chemical entity of cocrystal containing both nicotinamide riboside chloride and resveratrol has been discovered for the first time, and the preparation method is disclosed in the current patent.

Remarkably, the resveratrol cocrystal of nicotinamide riboside chloride has been identified with the molar ratio of the salt and the conformer spontaneously being towards to a stoichiometric relationship.

In some embodiments, the compound of Formula I is selected from the group consisting of:

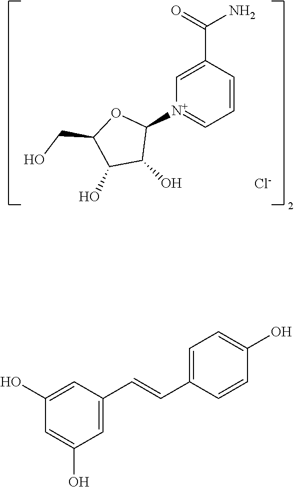

wherein X⁻is a chloride anion, the Coformer is resveratrol, m=2, n=1, which is abbreviated as [NR—Cl]₂ [RSV] cocrystal.

A resveratrol cocrystal of nicotinamide riboside chloride, sometimes also called as a nicotinamide riboside chloride resveratrol cocrystal or a nicotinamide riboside chloride cocrystal with resveratrol, has been discovered and prepared in acceptable yield. The chemical composition is characterized and confirmed by nuclear magnetic resonance (NMR) spectroscopy. Remarkably, resveratrol (RSV) cocrystal of nicotinamide riboside chloride (NR—Cl) crystallizes in an anhydrous form or a solvated form with an about 2:1 molar ratio of NR—Cl/RSV.

### Cocrystal Crystalline Forms

Remarkably, at least two different crystalline forms of the resveratrol cocrystal of nicotinamide riboside chloride with a 2:1 molar ratio of NR—Cl/RSV, namely Form A and Form B, have been discovered and analyzed. The crystalline material of each cocrystal form has been characterized by X-ray powder diffraction (XRPD) technique.

X-ray powder diffraction (XRPD) technique is regarded as the “golden tool” for identifying, analyzing, and/or characterizing crystalline forms. It also provides a fingerprint for each crystalline form with unique molecular conformation and molecular packing in its crystal lattice. The crystalline salts disclosed herein were characterized by X-ray powder diffraction technique and found to have unique XRPD patterns, respectively.

The term “powder X-ray diffraction pattern”, “X-ray powder diffraction pattern”, “PXRD pattern”, “XRPD pattern”, or “powder X-ray diffraction diagram” refers to the experimentally observed diffractogram or parameters derived therefrom. Powder X-ray diffraction patterns are characterized by peak positions (the horizontal or x-axis) and peak intensities (the vertical or y-axis). The term “2 theta value” or “2θ” refers to the peak position in degrees based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2 theta (2θ). The reference herein to specific 2θvalues for a specific solid form is intended to mean the 2θvalues (in degrees) as measured using the X-ray diffraction experimental conditions as described herein.

In one embodiment as disclosed herein, the exemplary crystalline form of [NR—Cl]₂[RSV] cocrystal is an anhydrous form, designated as Form A, and is characterized by a PXRD pattern comprising one or more peaks expressed as 2θ±0.2° of about 5.6°, about 11.0°, about 11.3°, about 12.3°, about 13.7°, about 14.0°, about 15.9°, about 19.8°, about 26.0°, and about 26.4°.

In another embodiment as disclosed herein, the exemplary crystalline form of [NR—Cl]₂[RSV] cocrystal is a solvated form, designated as Form B, and is characterized by a PXRD pattern comprising one or more peaks expressed as 20±0.2° of about 5.5°, about 8.2°, about 11.0°, about 12.1°, about 12.9°, about 13.5°, about 15.3°, about 19.6°, about 22.0°, and about 27.0°.

### Cocrystal Physicochemical Properties

Thermal stability is the stability of a molecule at elevated temperatures, and a molecule with more thermal stability has more resistance to decomposition at elevated temperatures. Thermal stability of a compound is one of the most important pharmaceutical properties that requires to be considered during developing a pharmaceutical or nutraceutical product. A better thermal stability of the drug substance at elevated temperature, which might also indicate a better physical stability and a better chemical stability in the ambient temperature, would benefit both the storage condition and the shelf life of the drug product. Differential Scanning calorimetry (DSC) is an analysis technique by measuring the heat change associated with the molecule's thermal events when heated at a constant rate. The temperature and its enthalpy change (usually the heat of fusion) at a specific thermal event recorded from DSC technique could be used to evaluate the thermal stability of an organic molecule directly. Higher melting/decomposition temperature and/or larger value of the heat of fusion suggests a more stable crystalline form because nicotinamide riboside starts to decompose at the melting point. It should be pointed out that NR—Cl-Form B with a higher melting/decomposing temperature (123° C.) with a larger value of heat of fusion (69 J/g) was found to be the most stable form among the three crystalline forms of nicotinamide riboside chloride identified so far. NR—Cl-Form A is a meta-stable form with a melting/decomposing temperature of 119° C. and a heat of fusion of 58 J/g. and NR—Cl-Form C is an unstable methanol solvate form, which started to de-solvate at a lower temperature. It is desirable to identify a new crystalline form with a higher melting/decomposition temperature and/or a larger heat of fusion to improve the thermal stability of the nicotinamide riboside drug substance.

Surprisingly, Form A of the resveratrol cocrystal of nicotinamide riboside chloride, [NR—Cl]₂[RSV]-Form A, shows a slightly higher melting/decomposing temperature and a much larger value of heat of fusion (124° C. and 100 J/g) compared to the most stable NR—Cl-Form B, suggesting [NR—Cl]₂[RSV]-Form A has better thermal stability than any of NR—Cl crystalline forms. [NR—Cl]₂[RSV]-Form B also shows a comparable melting/decomposing temperature to that of NR—Cl crystalline forms but a much larger value of heat of fusion (121° C. and 97 J/g). The improvement of the thermal stability by the resveratrol cocrystal would positively impact on the shelf life and the storage condition for the nicotinamide riboside products.

In addition to the thermal stability, the hygroscopicity, the tendency of a solid substance to absorb moisture from the surrounding atmosphere, is another important physicochemical property that needs to be considered during pharmaceutical development, because, like the heat, the moisture may also damage the drug product at a specific storage condition during the period of the drug shelf life. One of the key disadvantages of the nicotinamide riboside chloride products in the market is its poor hygroscopicity. As reported previously, all the three crystalline forms of the nicotinamide riboside chloride deliquesce at the relative humidity (RH) of 70%-80%. As a result, the nicotinamide riboside chloride requires to store in a dry place to avoid absorbing moisture from the air. A crystalline form with better hygroscopicity would mitigate the deliquescence risk when the nicotinamide riboside products are transported in a high humidity or stored in a high humidity environment, e.g., the bathroom of a house. The hygroscopicity assessment can be achieved by placing compounds in a specified relative humidity environment, e.g., ˜75% RH environment established with the sodium chloride saturated aqueous solution.

Surprisingly, both crystalline forms of the nicotinamide riboside chloride resveratrol cocrystal, [NR—Cl]₂[RSV], possess better hygroscopicity compared to crystalline forms of the nicotinamide riboside chloride itself. NR—Cl-Form A deliquesces after 1 day and NR—Cl-Form A deliquesces after 7 days in the environment of 75% RH, while both [NR—Cl]₂[RSV]-Form A and [NR—Cl]₂[RSV]-Form B retain as flowable crystalline powders at the same humidity environment for more than one month.

As disclosed herein, the resveratrol cocrystal of nicotinamide riboside chloride shows better thermal stability and hygroscopicity compared to the nicotinamide riboside chloride itself. These improved physicochemical properties would benefit the cocrystal form of nicotinamide riboside to be used as pharmaceutical or nutraceutical compositions in commercial drug products to treat or prevent cardiovascular disease, aging, or other conditions.

### Methods of Preparing Cocrystal of Nicotinamide Riboside Salt

In another aspect, provided herein is a method to prepare the desired cocrystal of nicotinamide riboside salt. The general reaction disclosed herein to prepare the [NR—Cl]₂[RSV] cocrystal by a reaction crystallization process is shown as the following scheme:

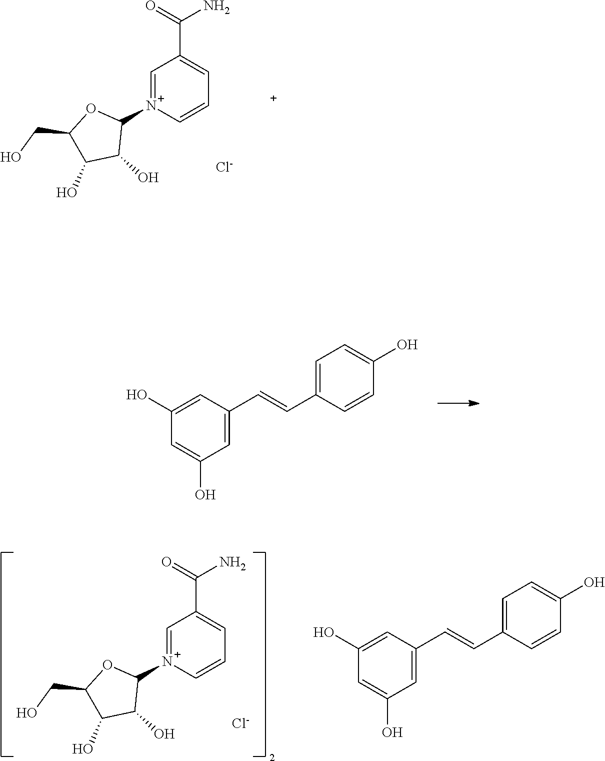

Reaction crystallization (or reactive crystallization or crystallization reaction) refers to the phenomenon comprising simultaneous reaction, mass transfer, crystal nucleation and growth, as well as possible secondary processes of aging, ripening, agglomeration and breakage. Reaction crystallization as disclosed herein is used in preparation of a cocrystal of nicotinamide riboside salt from a reaction mixture of the nicotinamide riboside salt and its coformer. In order to produce a high quality of the cocrystal of a salt from the reaction mixture, various factors, including the starting materials, crystallization methods, solvents, reaction temperatures and times, have been considered to achieve a robust reaction crystallization process.

Crystallization methods described herein includes the methods described in the Examples below, or by techniques including, but not limited to, heating, cooling, freeze drying, lyophilization, spray drying, quench cooling the melt, rapid solvent evaporation, slow solvent evaporation, solvent recrystallization, antisolvent addition, slurry recrystallization, crystallization from the melt, desolvation, recrystallization in confined spaces such as, e.g., in nanopores or capillaries, recrystallization on surfaces or templates such as, e.g., on polymers, recrystallization in the presence of additives, desolvation, dehydration, rapid cooling, slow cooling, exposure to solvent and/or water, drying, including, e.g., vacuum drying, vapor diffusion, sublimation, grinding (including, e.g., cryo-grinding and solvent-drop grinding), microwave-induced precipitation, sonication-induced precipitation, laser-induced precipitation and precipitation from a supercritical fluid. Unless otherwise specified, methods involving solvents described herein contemplate the use of any suitable common laboratory solvent, as known in the art. The particle size of resulting solid forms, which can vary, (e.g., from nanometer dimensions to millimeter dimensions), can be controlled, e.g.: by varying crystallization conditions (such as, e.g., the rate of crystallization and/or the crystallization solvent system); by altering spray drying operating parameters (including, e.g., feed solution concentration); and/or equipment design or by particle-size reduction techniques (e.g., grinding, milling, micronizing or sonication).

The preferred crystallization methods disclosed herein include, but are not limited to, recrystallization process, adding anti-solvent process, and slurry process. The recrystallization process includes at least two steps, including preparation of the cocrystal of a salt by evaporation of reaction solution containing the salt and cocrystal former (coformer), followed by stirring the resulting amorphous or hemi-crystalline form in a specific solvent to improve the crystallinity and chemical purity before collection of the solid. The anti-solvent process is dissolving starting materials in a solvent or mixed solvents, followed by precipitating the desired crystalline solid by adding the ani-solvent, in which the cocrystal of a salt has poor solubility. The slurry process is a method to stir the suspension of the reaction mixture until the cocrystal of a salt is formed, and then adding anti-solvent to improve the yield.

The term “solvent” as used herein refers to any inorganic or organic solvent. Certain solvents can be a part of the cocrystal of a salt, e.g., water, as disclosed herein. Solvents are useful in the disclosed method or article, product, or composition as reaction solvent or carrier solvent. Suitable solvents include, but are not limited to, lower alkyl alcohols, aliphatic and aromatic hydrocarbons, dialkyl ethers, dialkyl ketones, alkyl acetates, acetonitrile, chlorinated alkanes, dimethyl sulfoxide (DMSO), dimethylformamide (DMF), and aqueous solvents. Examples of solvents include, but are not limited to, methanol (MeOH), ethanol (EtOH), propanol, isopropanol (IPA) and butanol, isobutanol, ethyl acetate, iso-propyl acetate, n-heptane, diethyl ether, tert-butyl methyl ether, acetone, dichloromethane, or water. The solvents used herein can be of a single solvent or a mixture of many different solvents. The cocrystal of nicotinamide riboside salt was prepared through reaction crystallization with selection of reaction different solvents. The most commonly used solvents are water (H₂O) and alcohol. The alcohol examples herein include, without limitation, methanol (MeOH), ethanol (EtOH) and isopropanol (IPA).

Various solvents are selected based on solubility of starting materials and crystallization methods disclosed herein. The nicotinamide riboside chloride salt is soluble in water and MeOH but poorly soluble in EtOH and IPA. On the other hand, the resveratrol coformer is poorly soluble in water but soluble in alcohols of MeOH, EtOH and IPA. These differences in solubilities also impact the ratio of the two starting materials. In the disclosed methods to prepare crystalline berberine ascorbate, a ratio between the nicotinamide riboside chloride salt and resveratrol cofomer from about 2:1 to about 2:2 to prepare [NR—Cl]₂[RSV] with a NR—Cl/RSV molar ratio of 2:1.

Reaction crystallization temperatures and times depend upon targeted crystalline form of the cocrystal of a salt that is to be crystallized, the concentration of the salt and coformer in solution, and the solvent system. It should be noted that the elevated temperature or the long reaction time should be avoided to reduce the possibility of chemical decomposition for the nicotinamide ribose. It was observed at an elevated temperature above 50° C., the nicotinamide riboside chloride decomposed in alcohol suspension within 1 hour. Even in room temperature, the nicotinamide riboside chloride starts to decompose after stirring in the alcohol suspension for more than 3 days. The preferred reaction time is less than 24 hours with reaction temperature at around room temperature.

In one embodiment as disclosed herein, the disclosed method uses recrystallization process to prepare Form A of [NR—Cl]₂[RSV] cocrystal by the procedures. 1) Dissolving the starting materials of NR—Cl and RSV in MeOH, MeOH/H₂O, EtOH/H₂O or IPA/H₂O. 2) Concentrating the resulting solution in a vacuum to dryness. 3) Stirring the resulting solid in dry EtOH solvent. 4) Isolating the resulting crystalline Form A of [NR—Cl]₂[RSV] by filtration, washing and drying.

In one embodiment as disclosed herein, the disclosed method uses adding anti-solvent process to prepare Form B of [NR—Cl]₂[RSV] cocrystal by the procedures. 1) Dissolving the starting materials of NR—Cl and RSV in MeOH/H₂O, EtOH/H₂O or IPA/H₂O. 2) Adding anti-solvent of IPA into the solution and stirring to produce a homogenous slurry. 3) Isolating the resulting crystalline Form B of [NR—Cl]₂[RSV] by filtration, washing and drying.

In one embodiment as disclosed herein, the disclosed method uses slurry process to prepare Form B of [NR—Cl]₂[RSV] cocrystal by the procedures. 1) Suspending the starting materials of NR—Cl and RSV in minimum amount of EtOH/H₂O co-solvent. 2) Stirring the suspension until the starting physical mixture fully converted cocrystal to yield a wet-cake. 3) Adding EtOH into the resulting wet-cake and stirring to produce a homogenous slurry. 4) Isolating the resulting crystalline Form B of [NR—Cl]₂[RSV] by filtration, washing and drying.

### Pharmaceutical Compositions

A composition disclosed herein includes an effective amount or a therapeutically effective amount of a nicotinamide riboside cocrystal as disclosed herein.

As one skilled in the art will ascertain, an effective amount or an amount sufficient to treat (e.g., therapeutically effective amount) refers to the amount of a pharmaceutical composition administered to improve, inhibit, or ameliorate a condition of a subject, or a symptom of a disorder, in a clinically relevant manner. Any improvement in the subject is considered sufficient to achieve the treatment. Preferably, an amount sufficient to treat is an amount that prevents the occurrence or one or more symptoms of the diseases or conditions or is an amount that reduces the severity of, or the length of time during which a subject suffers from, one or more symptoms of the diseases or conditions.

By its “pharmaceutical composition”, the cocrystal of nicotinamide riboside salt as disclosed herein provides the therapeutically or biologically active agent for formulation into a suitable delivery means for administration to a subject. For the purposes of this disclosure, pharmaceutical compositions suitable for delivering the nicotinamide riboside as disclosed herein can include, e.g., tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of the aforementioned formulations can be prepared by well-known and accepted methods of art.

In an aspect, the pharmaceutical compositions disclosed herein comprise a cocrystal of nicotinamide riboside as disclosed herein and a pharmaceutically acceptable carrier or excipient. The term “pharmaceutically acceptable” means approved by a regulatory agency of the Federal or a state government or listed in the U.S. Pharmacopeia or other generally recognized pharmacopcia for use in animals, and more particularly in humans. Examples of suitable pharmaceutically acceptable carriers or excipients that can be used in said pharmaceutical compositions include, but are not limited to, sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, phosphate buffer solutions, lubricants, coloring agents, releasing agents, coating agents, sweetening, flavoring or perfuming agents, preservatives, or antioxidants.

The term “excipient” refers to additives and stabilizers typically employed in the art (all of which are termed “excipients”), including for example, buffering agents, stabilizing agents, preservatives, isotonifiers, non-ionic detergents, antioxidants and/or other miscellaneous additives. Stabilizers refer to a broad category of excipients which can range in function from a bulking agent to an additive which solubilizes the disclosed salts or helps to prevent denaturation of the same. Additional conventional excipients include, for example, fillers (e.g., starch), chelating agents (e.g., EDTA), antioxidants (e.g., ascorbic acid, methionine, vitamin E) and cosolvents.

The term “carrier” refers to a diluent, adjuvant, excipient, or vehicle with which the pharmaceutical composition is administered. Such pharmaceutical carriers are illustratively sterile liquids, such as water and oils, including those of petroleum, animal, vegetable, or synthetic origin, such as peanut oil, soybean oil, mineral oil, sesame oil and the like. Water is a preferred carrier when the pharmaceutical composition is administered intravenously. Saline solutions and aqueous dextrose and glycerol solutions are optionally employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, also contains wetting or emulsifying agents, or pH buffering agents. These compositions optionally take the form of solutions, suspensions, emulsion, tablets, pills, capsules, powders, sustained release formulations and the like. The composition is optionally formulated as a suppository, with traditional binders and carriers such as triglycerides. Oral formulation illustratively includes standard carriers such as pharmaceutical grades of mannitol, lactose, starch, magnesium stearate, sodium saccharine, cellulose, magnesium carbonate, etc.

In an aspect, pharmaceutical compositions according to the disclosure may be formulated to release the composition immediately upon administration (e.g., targeted delivery) or at any predetermined period after administration using controlled or extended-release formulations. Administration of the pharmaceutical composition in controlled or extended-release formulations is useful where the composition, either alone or in combination, has (i) a narrow therapeutic index; (ii) a narrow absorption window in the gastro-intestinal tract; or (iii) a short biological half-life, so that frequent dosing during a day is required in order to sustain a therapeutic level. One skilled in the art will ascertain compositions for controlled or extended release of the pharmaceutical composition. In an aspect, controlled release can be obtained by controlled release compositions and coatings which are known to those of skill in the art.

### Methods of Use/Treatment

A cocrystal of nicotinamide riboside salt as disclosed herein is employed in methods of therapeutic or prophylactic treatment of a subject, which may be referred to as an animal, including a human, to treat or prevent cardiovascular diseases, aging, or other conditions. As referred to herein, cardiovascular diseases, aging, or conditions include any disease states or conditions that can be or can be treated or prevented by the cocrystal of nicotinamide riboside salt or other medications.

The “treating” means administering the cocrystal of nicotinamide riboside salt as disclosed herein for prophylactic and/or therapeutic purposes. Prophylactic treatment may be administered, for example, to a subject who is not yet ill, but who is susceptible to, or otherwise at risk of, a particular disorder, e.g., cardiovascular diseases, aging, or other conditions. Prophylactic treatment reduces the likelihood of a subject developing cardiovascular diseases, aging, or conditions. Therapeutic treatment may be administered, for example, to a subject already suffering from a disorder to improve or stabilize the subject's condition. Thus, in the claims and embodiments described herein, treating is the administration to a subject either for therapeutic or prophylactic purposes.

The methods of treatment disclosed herein may be performed alone or in conjunction with another treatment. The methods of treatment may further be combined with other therapeutic agents.

### EXAMPLES

The present disclosure is further illustrated by the following examples, which should not be considered as limiting in any way. Embodiments of the present disclosure are further defined in the following non-limiting examples. It should be understood that these examples, while indicating certain embodiments of the disclosure, are given by way of illustration only. From the above discussion and these examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Chemicals

All the compounds, including nicotinamide riboside chloride and resveratrol used in the experiments, were purchased from Amazon and used without further purification.

### Analytical Methods

### Nuclear Magnetic Resonance (NMR) Spectroscopy

In general, the structures of end-products of the cocrystal of nicotinamide riboside salt were confirmed by nuclear magnetic resonance (NMR) spectroscopy. Proton magnetic resonance spectra were taken using a Bruker Advance 500 (500 MHz) and NMR chemical shift values were given in ppm. Proton NMR measurements were taken at ambient temperature unless otherwise specified; the following abbreviations have been used for characterizing NMR peaks: s, singlet; d, doublet; t, triplet; q, quartet; m, multiplet; dd, doublet of doublets; ddd, doublet of doublet of doublet; dt, doublet of triplets; bs, broad signal. The chemical shifts (δ) of the NMR peak are reported in parts per million (ppm) downfield of tetramethylsilane (TMS) and referenced to the respective residual un-deuterated solvent peak as follows: MeOH-d₄=3.31 ppm for ¹H-NMR. Apparent coupling constants (J) are reported in Hz.

### X-Ray Powder Diffraction (XRPD) Analysis

In general, the crystalline forms of the cocrystal of nicotinamide riboside salt were analyzed by X-ray powder diffraction (XRPD), which provides a fingerprint of a crystalline form. XRPD analysis was performed using a Bruker D8 diffractometer by Bruker AXS Inc™(Madison, Wisconsin). The XRPD spectra were obtained by mounting a sample (approximately 5-10 mg) of the material for analysis on a single silicon crystal wafer mount (e.g., a Bruker silicon zero background X-ray diffraction sample holder) and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms (e.g., about 1.54 angstroms). The sample was exposed for 1 second per 0.02 degree 2-theta increment (continuous scan mode) over the range 5 degrees to 40 degrees 2-theta in theta-theta mode. The running time was ˜15 min for such a measurement.

XRPD 2θ values may vary with a reasonable range, e.g., in the range ±0.2° and the XRPD intensities may vary when measured for essentially the same crystalline form for a variety of reasons including, for example, preferred orientation.

Differential Scanning calorimetry (DSC) Analysis

DSC analysis was performed on samples prepared according to standard methods using a Q SERIES™ Q2500 DSC calorimeter available from TA INSTRUMENTS® (New Castle, Delaware). A sample (approximately 2 mg) was weighed into an aluminum sample pan and transferred to the DSC. The instrument was purged with nitrogen at 50 mL/min and data collected between 22° C. and 300° C., using a dynamic heating rate of 10° C./minute. Thermal data was analyzed using standard software, e.g., TRIOS from TA INSTRUMENTS®.

### Thermogravimetry Analysis (TGA)

TGA was performed on samples prepared according to standard methods using a Q SERIES™ Q5500 thermogravimetry analyzer available from TA Instruments INSTRUMENTS® (New Castle, Delaware). A sample (approximately 5 mg) was placed into an aluminum sample pan and transferred to the TGA furnace. The instrument was purged with nitrogen at 50 mL/min and data collected between 25° C. and 300° C., using a dynamic heating rate of 10° C./minute. Thermal data was analyzed using standard software, e.g., TRIOS from TA INSTRUMENTS®.

### EXAMPLE 1

### Preparation and Characterization of an Exemplary Form A of Resveratrol Cocrystal of Nicotinamide Riboside Chloride

A light-yellow solution was obtained by dissolving 116 mg of NR—Cl (0.40 mmol) and 69 mg of resveratrol (0.30 mmol) in 2.0 ml of EtOH and 0.4 ml of water (H₂O). A light-yellow solid was obtained after the resulting solution was concentrated in vacuum to dryness. A suspension was obtained after 2.0 ml of EtOH was added to the solid. The suspension was stirred at room temperature for 2 hours to yield a homogeneous slurry. The creamy solid was filtered and dried in vacuum for 2 hours. 138 mg of [NR—Cl]₂[RSV]-Form A was obtained (Yield: 85%).

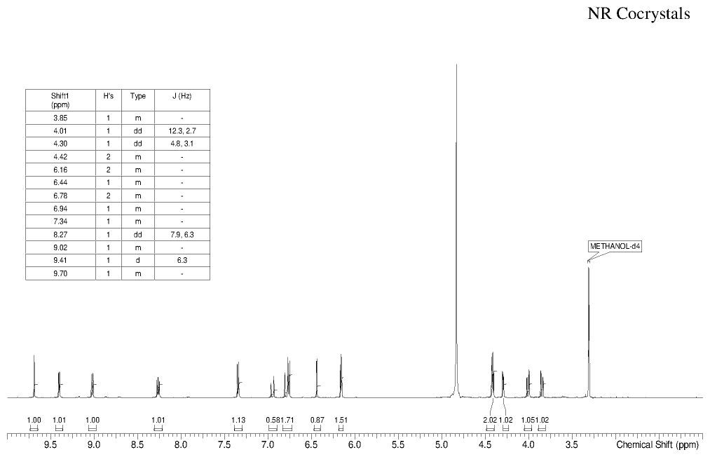

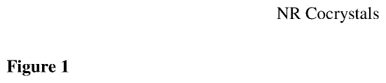

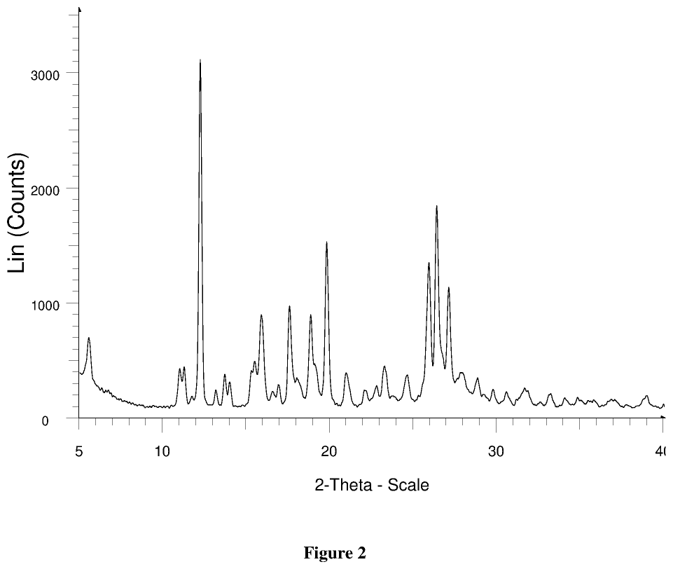

The proton NMR spectrum of [NR⁺Cl⁻]₂[Resveratrol] cocrystal Form A in CD₃OD, shown in FIG. 1, indicates an about 2:1 ratio of the nicotinamide riboside and the resveratrol for the cocrystal. ¹H NMR (500 MHZ, METHANOL-d₄) δ ppm 3.81-3.89 (m, 1 H) 4.01 (dd, J=12.28, 2.67 Hz, 1 H) 4.30 (dd, J=4.81, 3.13 Hz, 1 H) 4.37-4.46 (m, 2 H) 6.13-6.19 (m, 2 H) 6.40-6.47 (m, 1 H) 6.72-6.83 (m, 2 H) 6.90-6.99 (m, 1 H) 7.30-7.39 (m, 1 H) 8.27 (dd, J=7.86, 6.33 Hz, 1 H) 8.989.07 (m, 1 H) 9.41 (d, J=6.26 Hz, 1 H) 9.65-9.74 (m, 1 H).

[NR⁺Cl⁻]₂[Resveratrol] cocrystal Form A was analyzed by XRPD. The key peaks from the XRPD pattern are tabulated in Table 1 and the XRPD pattern is shown in FIG. 2.

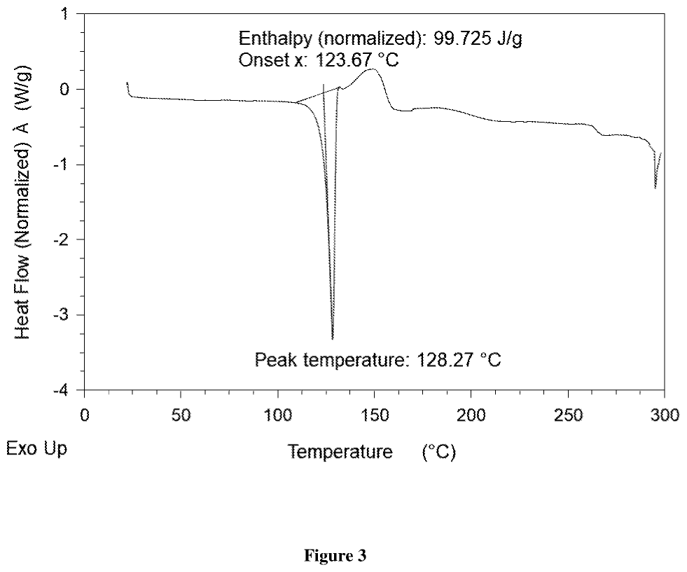

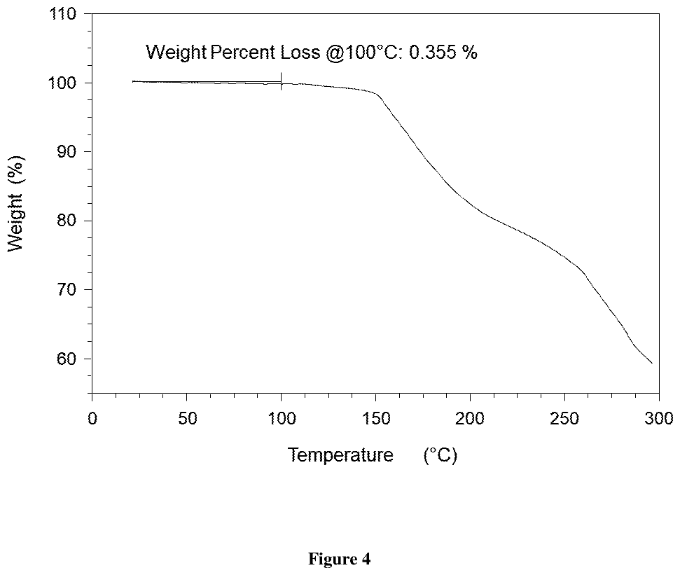

TABLE 1
XRPD Peaks for [NR—Cl]₂[RSV]-Form A

| Angle (2θ ± 0.2°) | Intensity (%) |
|---|---|
| 12.3 | 100.0 |
| 26.4 | 59.1 |
| 19.8 | 49.0 |
| 26.0 | 43.3 |
| 27.2 | 36.3 |
| 17.6 | 31.1 |
| 15.9 | 28.7 |
| 18.9 | 28.6 |
| 5.6 | 22.3 |
| 15.5 | 15.6 |
| 19.2 | 14.5 |
| 23.3 | 14.3 |
| 11.3 | 14.1 |
| 11.0 | 13.6 |
| 15.4 | 12.7 |
| 27.9 | 12.5 |
| 21.0 | 12.4 |
| 13.7 | 12.0 |
| 24.7 | 11.9 |
| 28.9 | 11.0 |
| 18.1 | 10.9 |
| 14.0 | 9.8 |
| 17.0 | 9.1 |
| 22.8 | 8.8 |
| 31.7 | 8.1 |
| 29.8 | 7.8 |
| 13.2 | 7.6 |
| 22.1 | 7.5 |
| 30.6 | 7.1 |
| 33.3 | 6.6 |
| 39.1 | 6.0 |
| 34.1 | 5.5 |

[NR—Cl]₂[RSV]-Form A was also analyzed by thermal analysis techniques. DSC analysis, shown in FIG. 3, indicates that Form A has an endotherm event of melting/decomposition with an onset at about 124° C. and a peak at about 128° C. TGA, shown in FIG. 4, indicates that Form A exhibits a mass loss of 0.4% upon heating from about 25° C. to about 100° C.

### EXAMPLE 2

### Preparation and Characterization of an Exemplary Form B of Resveratrol Cocrystal of Nicotinamide Riboside Chloride

Method 1. Preparation from Anti-Solvent Method

A light-yellow solution was obtained by dissolving 58 mg of NR—Cl (0.20 mmol) and 25 mg (0.11 mmol) in 0.5 ml of isopropanol (IPA) and 0.2 ml of H₂O. To the clear solution, 1.5 ml of IPA was added slowly, and a cloudy light-yellow solution was obtained. The resulting cloudy solution was stirred at the room temperature for 1 day, a homogenous slurry was obtained. The creamy solid was filtered and dried in vacuum for 2 hours. 48 mg of [NR—Cl]₂[RSV]-Form B was obtained (Yield: 59%).

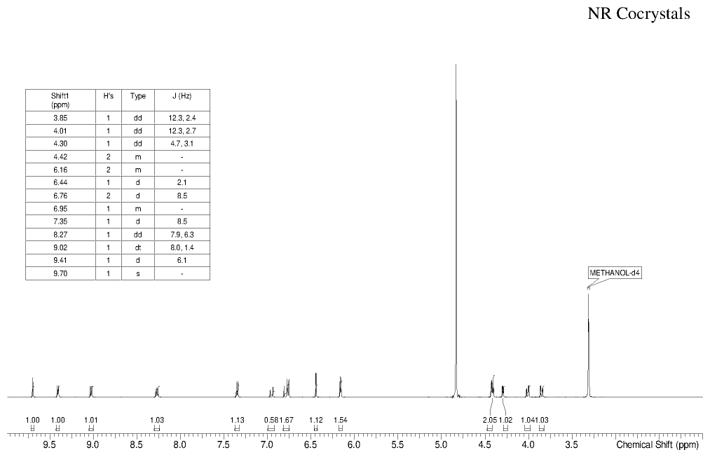

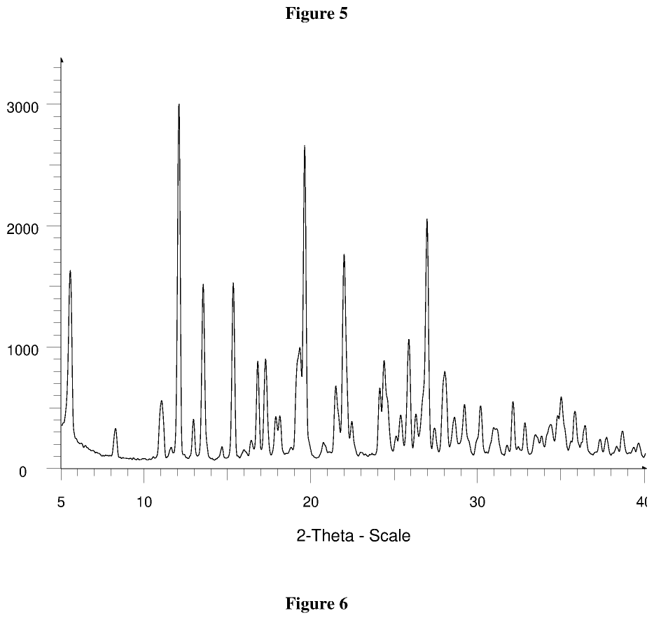

The proton NMR spectrum of [NR—Cl]₂[RSV]-Form B in CD₃OD, shown in FIG. 5, indicates an about 2:1 ratio of the nicotinamide riboside and the resveratrol for the cocrystal. ¹H NMR (500 MHZ, METHANOL-d₄) δ ppm 3.85 (dd, J=12.28, 2.37 Hz, 1 H) 4.01 (dd, J=12.28, 2.67 Hz, 1 H) 4.30 (dd, J=4.73, 3.05 Hz, 1 H) 4.39-4.45 (m, 2 H) 6.14 - 6.18 (m, 2 H) 6.44 (d, J=2.14 Hz, 1 H) 6.76 (d, J=8.54 Hz, 2 H) 6.91-7.00 (m, 1 H) 7.35 (d, J=8.54 Hz, 1 H) 8.27 (dd, J=7.86, 6.33 Hz, 1 H) 9.02 (dt, J=8.05, 1.39 Hz, 1 H) 9.41 (d, J=6.10 Hz, 1 H) 9.70 (s, 1 H).

[NR—Cl]₂[RSV]-Form B was analyzed by XRPD. The key peaks from the XRPD pattern are tabulated in Table 2 and the XRPD pattern is shown in FIG. 6.

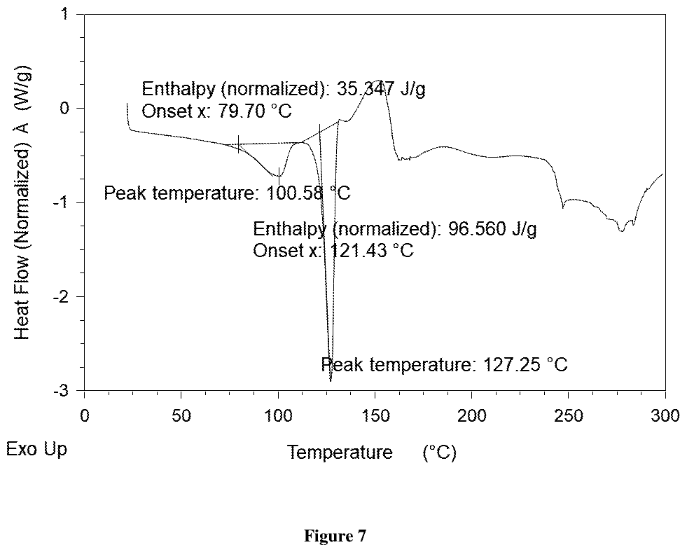

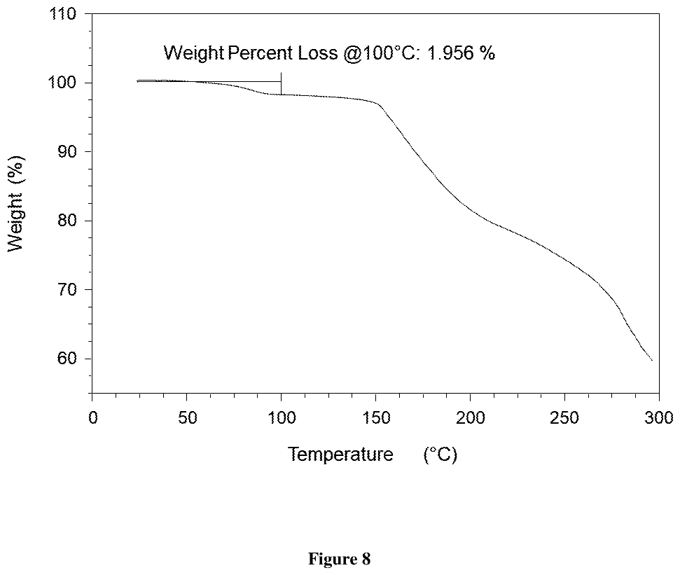

TABLE 2
XRPD Peaks for [NR—Cl]₂[RSV]-Form B

| Angle (2θ ± 0.2°) | Intensity (%) |
|---|---|
| 12.1 | 100.0 |
| 19.6 | 88.5 |
| 27.0 | 68.4 |
| 22.0 | 58.7 |
| 5.5 | 54.2 |
| 15.3 | 50.8 |
| 13.5 | 50.5 |
| 25.9 | 35.3 |
| 19.3 | 33.1 |
| 17.3 | 29.9 |
| 24.4 | 29.4 |
| 16.8 | 29.3 |
| 28.0 | 26.5 |
| 21.5 | 22.5 |
| 24.1 | 21.9 |
| 35.1 | 19.4 |
| 11.0 | 18.5 |
| 32.1 | 18.1 |
| 29.2 | 17.4 |
| 30.2 | 17.0 |
| 35.9 | 15.5 |
| 26.3 | 14.7 |
| 25.4 | 14.5 |
| 18.1 | 14.3 |
| 34.8 | 14.3 |
| 17.9 | 14.0 |
| 28.6 | 13.9 |
| 12.9 | 13.3 |
| 22.4 | 12.8 |
| 32.9 | 12.4 |
| 34.4 | 11.9 |
| 36.5 | 11.6 |
| 27.4 | 10.9 |
| 8.2 | 10.8 |
| 31.2 | 10.7 |
| 38.7 | 10.0 |
| 33.5 | 9.1 |
| 25.1 | 8.7 |
| 37.8 | 8.4 |
| 37.4 | 7.8 |
| 16.4 | 7.6 |
| 20.8 | 7.0 |
| 39.7 | 6.8 |
| 31.8 | 6.2 |
| 14.6 | 5.8 |
| 11.6 | 5.7 |

[NR—Cl]₂[RSV]-Form B was also analyzed by thermal analysis techniques. DSC analysis, shown in FIG. 7, indicates that Form B has an endotherm event of de-solvation with an onset at about 80° C. and a peak at about 101° C., followed by an endotherm event of melting/decomposition with an onset at about 121° C. and a peak at about 127° C. TGA, shown in FIG. 8, indicates that Form B exhibits a mass loss of 2.0% upon heating from about 25° C. to about 100° C.

Method 2. Preparation from Slurry Method

A light-yellow slurry was obtained by mixing 58 mg of NR—Cl (0.20 mmol) and 28 mg (0.12 mmol) in 0.50 ml of ethanol (EtOH) and 0.05 ml of H₂O. A light-yellow wet cake was obtained after stirring the slurry at room temperature for 1 day. 1.0 ml of EtOH was added to the wet cake, and a homogenous slurry was obtained after stirring at room temperature for 1 hour. The creamy solid was filtered and dried in vacuum for 2 hours. 42 mg of [NR—Cl]₂[RSV]-Form B was obtained (Yield: 52%).

### EXAMPLE 3

### Hygroscopicity Evaluation Study of [NR—Cl]₂[RSV] cocrystals and NR—Cl salts

A Kerr SureTight™ container of 8 oz Clear Glass Wide Mouth Mason Jars with Silver Vacuum Seal Lid was used to secure the closure. An 8 ml opened vial containing sodium chloride (NaCl) saturated aqueous solution (about 75.5% RH at 20° C. and about 75.3% at 25° C. from www.engineeringtoolbox.com/salt-humidity-d_1887.html) was placed in the SureTight container. The 4 ml of opened vials containing about 5 mg of [NR—Cl]₂[RSV] cocrystals or NR—Cl crystalline forms were also placed in the same container with the vial of the NaCl saturated aqueous solution. The container was sealed with the seal lid and placed in a room temperature condition (˜22° C.). The powder property was visually observed directly or during shaking the vial. Crystallinity was assessed under a polarized light microscope.

After 1 day, white powder of NR—Cl-Form A deliquesced. White powder of NR—Cl-Form A became sticky and could not flow freely. Creamy powders of [NR—Cl]₂[RSV]-Form A and [NR—Cl]₂[RSV]-Form B remains as flowable powders.

After 7 days, the sticky powder of NR—Cl-Form A deliquesced. Creamy powders of [NR—Cl]₂[RSV]-Form A and [NR—Cl]₂[RSV]-Form B remains as flowable powders. After 30 days, both NR—Cl-Form A and NR—Cl-Form B are glass-like liquids (amorphous materials). Creamy powders of [NR—Cl]₂[RSV]-Form A and [NR—Cl]₂[RSV]-Form B remains as flowable crystalline powders.

After 30 days, both NR—Cl-Form A and NR—Cl-Form B are glass-like liquids (amorphous materials). Creamy powders of [NR—Cl]₂[RSV]-Form A and [NR—Cl]₂[RSV]-Form B remains as flowable crystalline powders.

What is claimed is:

1. A cocrystal of a nicotinamide riboside salt has one of the following structures: 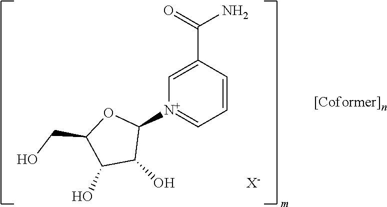
wherein X⁻is a negatively charged anion including but not limited to chloride, bromide, sulfate, phosphate, methyl sulfonate, acetate, malate, tartrate, citrate, fumarate, lactate, maleate, mandelate, malonate, succinate, ascorbate, gluconate, benzoate, besylate, tosylate, hippurate, camsylate, napasylate, napadisylate, stearate, undecylenate, and other acidic counterions; wherein the Coformer (cocrystal former) is a neutral compound or a neutral form of an acid or base, which is selected from any drug excipients included in the GRAS (Generally Recognized as Safe) list published by the FDA; wherein m and n are integers larger than 1; wherein the cocrystal is of anhydrous form, hydrate, solvate, or a mixture thereof; wherein the cocrystal is of a single crystalline form, or a mixture of multiple crystalline forms.

2. The cocrystal of nicotinamide riboside salt according to claim 1, wherein X⁻is the chloride anion, the Coformer is resveratrol; wherein the cocrystal is of anhydrous form, hydrate, solvate, or a mixture thereof, wherein the cocrystal has one of the following structures: 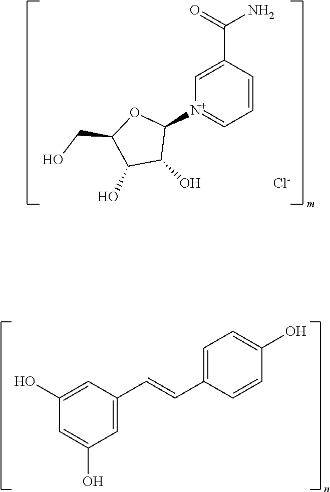
wherein m and n are integers larger than 1.

3. The resveratrol cocrystal of the nicotinamide riboside chloride according to claim 2, wherein m=2, n=1; wherein the cocrystal is of a single crystalline form, or a mixture of multiple crystalline forms; wherein the cocrystal has the following chemical structure.

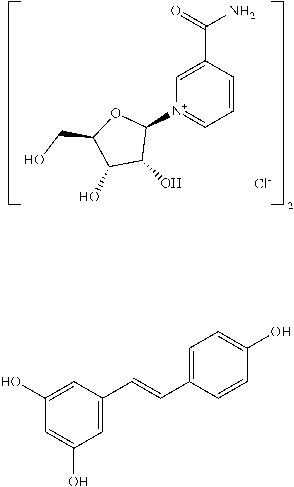

4. The resveratrol cocrystal of nicotinamide riboside chloride according to claim 3, wherein the cocrystal is an anhydrous form, designated as Form A, and is characterized by a PXRD pattern comprising one or more peaks expressed as 2θ±0.2° of about 5.6°, about 11.0°, about 11.3°, about 12.3°, about 13.7°, about 14.0°, about 15.9°, about 19.8°, about 26.0°, and about 26.4;, wherein the PXRD pattern of Form A is shown in FIG. 2.

5. The resveratrol cocrystal of nicotinamide riboside chloride according to claim 3, wherein the cocrystal is a solvated form, designated as Form B, and is characterized by a PXRD pattern comprising one or more peaks expressed as 2θ±0.2° of about 5.5°, about 8.2°, about 11.0°, about 12.1°, about 12.9°, about 13.5°, about 15.3°, about 19.6°, about 22.0°, and about 27.0°; wherein the PXRD pattern of Form B is shown in FIG. 6.

6. A method of preparing the resveratrol cocrystal of nicotinamide riboside chloride according to claim 3 by using recrystallization process. 1) Dissolving the starting materials of nicotinamide riboside chloride and resveratrol in alcohol, water, or their combinations. 2) Concentrating the resulting solution in a vacuum to dryness. 3) Stirring the resulting solid in a dry alcohol solvent. 4) Isolating the resulting crystalline material by filtration, washing, and drying.

7. A method of preparing the resveratrol cocrystal of nicotinamide riboside chloride according to claim 3 by using adding anti-solvent process. 1) Dissolving the starting materials of nicotinamide riboside chloride and resveratrol in alcohol, water, or their combinations. 2) Adding anti-solvent of an alcohol into the solution and stirring to produce a homogenous slurry. 3) Isolating the resulting crystalline material by filtration, washing, and drying.

8. A method of preparing the resveratrol cocrystal of nicotinamide riboside chloride according to claim 3 by using slurry process. 1) Suspending the starting materials of nicotinamide riboside chloride and resveratrol in a minimum amount of alcohol/water co-solvent. 2) Stirring the suspension until the starting physical mixture fully converted cocrystal to yield a wet-cake. 3) Adding anti-solvent of an alcohol into the resulting wet-cake and stirring to produce a homogenous slurry. 4) Isolating the resulting crystalline material by filtration, washing, and drying.

9. A composition comprising a cocrystal of a nicotinamide riboside salt according to claim 1 and a pharmaceutically or nutraceutically acceptable carrier.

10. The composition according to claim 9, wherein the carrier is a diluent, adjuvant, excipient, vehicle, or mixture thereof.

11. The composition according to claim 9, wherein the composition is formulated into tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols.

12. The composition according to claim 9, wherein the composition is an immediate release composition or extended-release composition.

13. A method of therapeutic or prophylactic treatment of a subject against central nervous system diseases, cardiovascular diseases, infection diseases, aging, or conditions comprising administration of an amount of a cocrystal of a nicotinamide riboside salt according to claim 1 to a subject in need of central nervous system diseases, cardiovascular diseases, infection diseases, or conditions care.

14. A method of therapeutic or prophylactic prevention of a subject against aging, poor mental conditions, poor cardiovascular conditions, or conditions comprising administration of an amount of a cocrystal of a nicotinamide riboside salt according to claim 1 to a subject in need of anti-aging, longevity, and improvement of mental conditions, cardiovascular conditions, or other conditions.
